(19) United States
(12) Patent Application Publication
(10) Pub. No.: US 2009/0281419 A1
(43) Pub. Date: Nov. 12, 2009

(54) System for determining the position of a medical instrument

(76) Inventors: Volker Troesken, Witten (DE); Laszlo Hasenau, Bochum (DE); Dietrich Groenemeyer, Sprockhoevel (DE)

Correspondence Address: COLLARD & ROE, P.C., 1077 NORTHERN BOULEVARD, ROSLYN, NY, 11576, US

(21) Appl. No.: 12/308,721

(22) PCT Filed: Jun. 22, 2007
(86) PCT No.: PCT/EP2007/005520

§ 371 Date: Mar. 20, 2009

(30) Foreign Application Priority Data
Jun. 22, 2006 (DE) 10 2006 029 122.0

(51) Int. Cl. A61B 5/05; G01S 3/02; G06K 19/06

(52) U.S. Cl. 600/424; 342/450; 235/492

(57) ABSTRACT

The invention relates to a system for determining the spatial position and/or orientation of a medical instrument (1), comprising a transmission unit (3) for transmitting electromagnetic radiation (4), at least one localisation element (2) that is arranged on the medical instrument (1) and which captures the electromagnetic radiation (4) transmitted by the transmission unit (3) and produces a localisation signal (5), and an evaluation unit (9) which determines the position and/or orientation of the medical instrument (1) by evaluating the localisation signal (5). The invention is characterised in that the localisation element (2) has a transponder that comprises an antenna (13) and a circuit (12) that is connected to the antenna (13). The circuit (12) can be excited by the electromagnetic radiation (4) of the transmission unit (3) captured by the antenna (13), such that the transmission unit emits, via the antenna (13), the localisation signal (5) as electromagnetic radiation.

The invention relates to a system for determining the spatial position and/or orientation of a medical instrument, comprising a transmission unit for transmitting electromagnetic radiation, at least one localisation element that is arranged on the medical instrument and which captures the electromagnetic radiation transmitted by the transmission unit and produces a localisation signal, and an evaluation unit which determines the position and/or orientation of the medical instrument by evaluating the localisation signal.

In medical science a precise determination of the position of an applied medical instrument is of paramount importance in various diagnostic and therapeutic is methods. Instruments of this kind, for example, may be intravascular catheters, guidance wires, biopsy needles, minimally invasive surgical instruments or the like. Those systems being of a particular interest are systems for determining the spatial position and location of a medical instrument in the field of interventional radiology.

For example, a system of the kind outlined hereinabove is known from EP 0 655 138 B1. With the prior art system, several transmission units are implemented which are spatially spread at defined positions. The transmission units transmit an electromagnetic radiation, possibly at a different frequency. To localize the medical instrument, a localisation element in form of a sensor receiving the electromagnetic radiation transmitted from the transmission units is arranged at this instrument. The sensor detects the electromagnetic field generated by the transmission units. The localization signal generated by the sensor corresponds to the electromagnetic field intensity at the site of the sensor and thus at the site of the medical instrument where the sensor is arranged. The localization signal is passed on to an evaluation unit. From the localization signal, the evaluation unit computes the sensor's distance to various transmission units. Since the transmission units are spatially spread at defined positions, the evaluation unit is capable of deriving the position of the medical instrument within the space based on the distances of the localization element from various transmission units.

The prior art system bears a disadvantage in that the localisation element is linked through a cable to the evaluation unit. The signal reflecting the field intensity of the electromagnetic radiation at the site of the localisation element is passed on through the cable to the evaluation unit. To localise medical instruments for minimally invasive interventions, in particular, cable connections of this kind are highly disadvantageous. Fitting electrical leads and plugged connections to minimally invasive instruments is extensive and expensive. Moreover, electrical feeder mains interfere on handling the instruments.

Against this background, it is an object of the present invention to provide an improved system to determine the spatial position and/or orientation of a medical instrument. Above all, the system should work without cable connections between the localisation element and the evaluation unit.

The present invention solves this task based on a system of the afore-mentioned kind in that the localisation element is comprised of a transponder having an antenna and a circuit connected to the antenna to receive and transmit electromagnetic radiation, with it being possible to excite said circuit by electromagnetic radiation from the transmission unit received via said antenna in such a manner that it transmits the localisation signal as an electromagnetic radiation via the antenna.

The key idea of the present invention is providing a medical instrument with a transponder which, for example, is utilized in well known RFID tags. The transponder antenna receives the electromagnetic radiation emitted from the transmission unit and thereby it itself is excited to transmit electromagnetic radiation. The transponder thus transmits the localisation signal as electromagnetic radiation without any cable connection. From the localisation signal radiated from the transponder, the evaluation unit determines the spatial position and/or orientation of the medical instrument.

It is of advantage that the localisation element of the inventive system can be produced at very low cost, because RFID tags are mass products that can be adapted at low expenditure to be suitable for the inventive application. Very small RFID transponders can be obtained commercially already now. The antenna of the transponder can be wound from a thin wire as a coil for integration into a medical instrument, with it being possible to arbitrarily adapt the coiling direction and geometry of the coil to the shape and size of a medical instrument.

With the inventive system, the transponder of the localisation element works in the same manner as known RFID transponders. The transmission unit generates a (high-frequency) electromagnetic field which is received by the antenna of the transponder. An inductive current is created in the antenna coil. It activates the circuit of the transponder. Once the circuit is activated, it transmits (high-frequency) electromagnetic radiation on the one hand, for example by modulating the field radiated from the transmission unit (by load modulation). Owing to the modulation, the electromagnetic radiation transmitted from the transponder lies within a side range of the radiation from the transmission unit. On this side range, the localisation signal is transmitted without any cable connection, i.e. wireless, to the evaluation unit for determining the position.

The transponder of the inventive system may be configured as a passive transponder, the electric power supply to the circuit being provided through the inductive current generated in the antenna on receipt of the electromagnetic radiation transmitted from the transmission unit. This embodiment of the inventive system bears the advantage in that the transponder works without an active energy supply of its own. The energy which the transponder requires to transmit the localisation signal is supplied by the electromagnetic field generated by the transmission unit. The transponder is expediently comprised of a capacitor for power supply to the circuit which is recharged by the inductive current generated in the antenna. The capacitor provides for a permanent supply of energy to the circuit. To recharge the capacitor, the medical instrument can be brought near to the transmission unit where the electromagnetic field generated by the transmission unit is adequately strong. As soon as the capacitor has been charged, the transponder works for a certain period of time also at a larger distance from the transmission unit. Since the supply of energy is ensured through the capacitor, the antenna of the transponder can be of a very small dimension, thus facilitating its integration into a medical instrument.

Alternatively, with the inventive system, the transponder may be configured as an active transponder, a battery being provided to supply power to the circuit. The transponder is activated expediently at the beginning of a medical intervention, for example when opening a packaging of a medical instrument. Alternatively, the circuit of the transponder is so configured that the supply of energy by the battery is not activated until the electromagnetic radiation transmitted from the transmission unit is activated.

In accordance with a purposive configuration of the inventive system, the frequency of the electromagnetic radiation of the localisation signal is different to the frequency of the electromagnetic radiation emitted from the transmission unit. It is thereby possible to differentiate the localisation signal transmitted from the transponder from the electromagnetic field generated by the transmission unit based upon the frequency. This can be realized as described hereinabove by the fact that the transponder generates the localisation signal by modulating the electromagnetic radiation emitted from the transmission unit. The frequency of the localisation signal then lies within a side range of the frequency of the electromagnetic radiation emitted by the transmission unit.

In accordance with an advantageous embodiment of the inventive system, the evaluation unit is connected at least to one receiver unit. It is conceivable to utilize several receiver units which receive the localisation signal transmitted by the transponder. Based upon the field intensity of the localisation signal at the site of the relevant receiver unit, one can derive the distance of the transponder from the receiver unit. If the distances of the transponder to various receiver units located at defined positions within the space are known, the precise position of the transponder and thus of the medical instrument within the space can be computed thereof by means of the evaluation unit.

It is problematic, however, that the field intensity of the localisation signal is attenuated if the medical instrument is introduced into a patient's body during an intervention. On account of its dielectric properties, body tissue partly absorbs the electromagnetic radiation transmitted from the transponder. For this reason, a determination of the position based upon the field intensity of the localisation signal cannot always be achieved with adequate accuracy.

To solve this problem the evaluation unit for determining the position and/or orientation of a medical instrument based on the phase relation of the electromagnetic radiation of the localisation signal can be provided at the relevant site of the receiver unit. With an appropriate choice of the localisation signal frequency, the influence of the dielectric properties of body tissue on the phase of the localisation signal is negligible. The transponder should be so equipped that it transmits the localisation signal coherently, i.e. with a defined and constant phase relation.

If the determination of position is made based on the phase relation of the electromagnetic radiation of the localisation signal as described hereinabove, it should be taken into account that a clear-cut allocation of a phase value to a position within space is possible only within a distance from the localisation element which is less than the wavelength of the localisation signal. With larger distances, it is additionally required to determine the zero crossings of the electromagnetic radiation of the localisation signal between the localisation element and the relevant receiving unit.

To achieve the highest possible accuracy in position determination it is purposive to use a circuit for the transponder of the localisation element with the inventive system that is provided at two or more different frequencies to generate the localisation signal. By generating the localisation signal at low frequencies and correspondingly large wavelengths, it is initially possible to obtain a rough though unambiguous determination of the position. To increase accuracy in position determination, a higher frequency is then chosen or the frequency of the localisation signal is successively incremented. With higher frequencies, requirements exacted from resolution in determining the phase relation to obtain a certain spatial resolution are lower. If the frequency is successively incremented, the number of zero crossings for determining the exact distance between localisation element and receiver unit can be determined. For a most accurate possible position determination, a frequency change in both directions, i.e. from low to high frequencies or from high to low frequencies is conceivable. Depending on the frequency ranges which have to be covered for position determination it might be required to provide two antennae or more which are connected to the circuit of the transponder, each of these antennae being allocated to a certain frequency range.

In accordance with a purposive embodiment of the inventive system, the transponder is connected at least to one sensor element, with the circuit of the transponder being so equipped that it transmits the sensor signal of the sensor element as an electromagnetic radiation via the antenna of the transponder. Accordingly, the transponder is not only utilized for position determination but also for transmission of sensor signals. The transponder is connected with appropriate sensor elements, for example a temperature sensor, a pressure sensor, a pH sensor or with a conventional position sensor. The transponder transmits the sensor signal in wireless mode as an analogue or digital signal.

The efficiency of the inventive system can be further increased by at least one additional localisation element which is not arranged at the medical instrument, said element being equipped with a transponder which is allocated to it and which can be detachably affixed a patient's body. For example, the additional localisation element can be detachably affixed by means of a glued, adhesive or a suction disk connection on a patient's skin surface. In accordance with a particularly practical configuration, the transponder of the additional localisation element is integrated into a self-adhesive foil or tissue strip like in a conventional plaster. By means of the additional localisation element, the position of a patient and/or of a certain part of a patient's body being of interest can be directly related to the position of the medical instrument. This is particularly advantageous for applications in interventional radiology. By way of the additional localisation element it is moreover made possible to consider a patient's body movements in positioning the medical instrument. For example, a patient's respiratory movement can be compensated for automatically in order to substantially improve accuracy of needle positioning in pulmonary biopsies. Another application becomes evident in the treatment of coronaries with an instrument (catheter) devised in the sense of the present invention in order to compensate for the heart muscle movement prompted by breathing. Hence, another aspect of the present invention is using an RFID tag for integration into a self-adhesive foil or tissue strip for detachable fixing on a patient's skin surface.

The inventive system can be used advantageously for position determination on MR-guided surgical interventions. The high-frequency transmission unit of the system which in any case does exist can purposively be utilized as transmission unit of the system. It comprises a transmission/receiver antenna, e.g. a body coil in form of a squirrel-cage resonator to generate a high-frequency electromagnetic field within the investigation volume of the MR appliance. As is well known, core-magnetic resonances in the body of an examined patient are excited by such an HF field on MR imaging. In this case, the transponder can practically be configured as a passive transponder, with the electric power supply to the circuit of the transponder being provided through the induction current generated on receipt of the HF field during MR imaging in the transponder antenna. Accordingly, the existing HF field in the MR appliance is exploited to supply energy to the transponder. In accordance with a purposive embodiment of the system, the evaluation unit can be connected to and/or integrated into the MR appliance, with the determination of the position and/or orientation of the medical instrument being performed based upon the localisation signal received through the transmission/receiver antenna of the MR appliance. Hence, with this configuration, the transmission/receiver antenna of the MR device is utilized for receiving the localisation signal. The localisation signal is transmitted via the receiver electronics of the MR device to the evaluation unit. It is particularly purposive, as has been outlined hereinabove, to determine the position of the localisation element based on the phase relation of the localisation signal. Accordingly, the evaluation unit linked to the MR device can advantageously be properly equipped to determine the position and/or orientation of a medical instrument based on the phase relation of the electromagnetic radiation of the localisation signal at the site of the transmission/receiver antenna of the MR device. The site of the transmission/receiver antenna is known and invariable. Therefore, this site can be taken as reference point in position determination based on the phase relation.

In medical technology systems the paramount goal is to achieve a failsafe operation. To this effect the inventive system can be so configured that the evaluation unit (like a so-called “voter”) can be properly equipped to select valid position and/or orientation data from a multiplicity of position and/or orientation data from several redundantly determined localisation signals. Accordingly, redundant position and/or orientation data are initially determined from localisation signals, for example by picking-up localisation signals repeatedly within short intervals or by picking-up localisation signals in parallel from several transponders arranged at a medical instrument. These redundant data are evaluated, compared to each other and/or checked for plausibility. Based on the outcome of this check-up, those position and/or orientation data recognised as valid data, i.e. applicable data, are selected. For example, it is possible to choose those position and/or orientation data which evidence more or less congruency with other redundantly determined data, while obviously diverging data (outliers) are recognized as faulty data and rejected. The localisation element may be comprised of a plurality of transponders, as has been outlined hereinabove, which can be excited in parallel and/or consecutively for transmission of localisation signals. It bears the advantage that a failsafe operation is ensured even in case individual transponders fail to work or their signals are not received or received in distorted mode (e.g. due to interference signals from the environment). This may also be achieved by arranging several localisation elements each of them comprised of one transponder or more at a medical instrument to generate redundant localisation signals. Redundancies in the sense of a higher fault-safety can be created, for example, by rating the transponders properly to generate localisation signals at different frequencies each. Interferences within individual frequency ranges will then not adversely affect a safe operation of the system.

The invention not only relates to a system for determining the position, but also to a medical instrument which is equipped with a transponder of the a.m. kind, as well as to a method for determining the spatial position and/or orientation of a medical instrument.

The key idea of the invention is to equip a medical instrument, e.g. an intravascular catheter, a guidance wire or a biopsy needle with an active or passive RFID tag of a conventional type in order to thus enable determining the spatial position and/or orientation of a medical instrument, preferably based upon the phase relation of the localisation signal generated by the RFID tag at a stationary site of reception. Moreover, the RFID tag can be utilized for wireless transmission of sensor signals from a sensor element also integrated into the medical instrument. The use of an RFID tag in a medical implant is also conceivable in order to be able to pick-up sensor signals, e.g. temperature, pressure, pH-value or even position signals from the site of implantation at any time.

It makes sense for the inventive transponder to comprise a data memory to save identification data, with the circuit for transmission of the identification data being properly equipped to transmit the identification data as an electromagnetic radiation via the antenna. Conventional RFID tags are comprised of such a digital data memory. The identification data can be utilized to differentiate various localisation elements in determining the spatial position and/or orientation from each other. For example, if it is intended to determine the orientation of a medical instrument, i.e. its position within space, it is expedient to equip the instrument with at least two inventively designed localisation elements. Based on the positions of the two localisation elements, one can derive the orientation of the instrument. The prerequisite to be fulfilled is that an identification of various localisation elements is possible, for example to be able to differentiate a localisation element arranged at the tip of a biopsy needle from a localisation element arranged at its handle component. Moreover, identification of the localisation elements is purposive if several medical instruments are applied in an intervention, because hazardous confusion in position determination can thus be avoided.

As has already been mentioned hereinabove, the inventive system can be utilized in combination with an imaging diagnostic device, for example a computerized tomography or an MR device, in order to allow for navigating with the applied interventional instrument. The position and orientation data determined by means of the inventive system can be visualized jointly with the imaged anatomic structures in order to make it easier for the physician performing the intervention to guide the instrument. It is an advantage that determining the spatial position and/or orientation with the inventive system is feasible independently of the imaging system. Thus it is possible to reduce radiation exposure during minimally invasive interventions. For the exact positioning and navigation of the instruments is feasible without a continuous radioscopy.

Examples of embodiments of the inventions are outlined in the following by way of drawings, wherein:

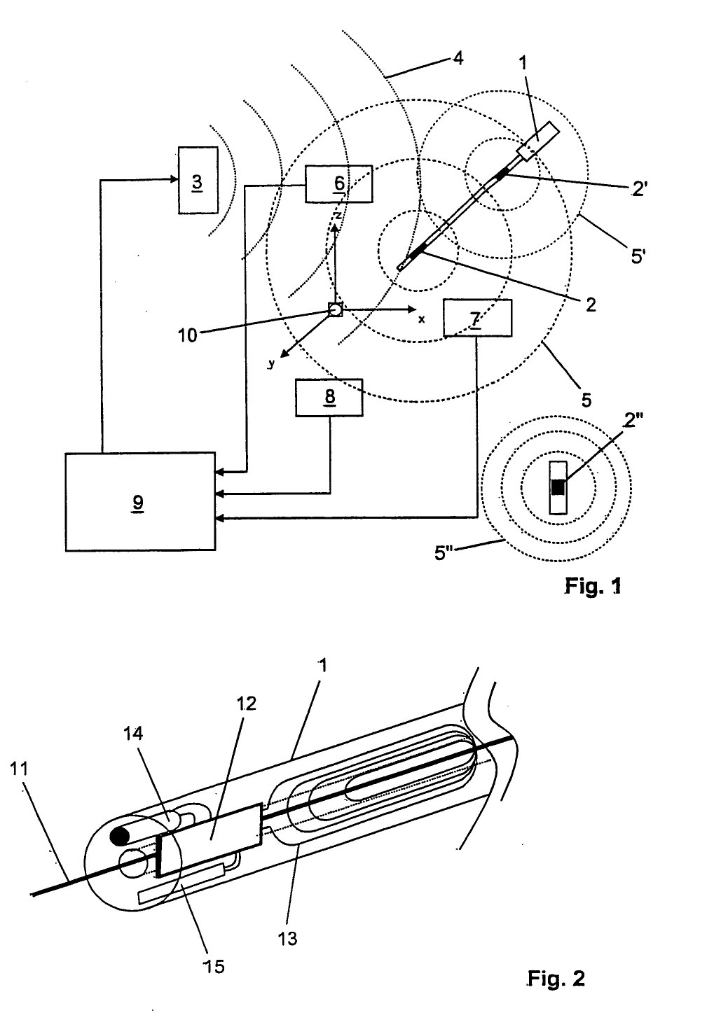

FIG. 1 shows the inventive system as a block diagram;

FIG. 2 is a schematic representation of an inventively configured medical instrument.

The system shown in FIG. 1 serves to determine the spatial position and orientation of a medical instrument 1. Arranged at the medical instrument 1 are localisation elements 2 and 2′. The system is comprised of a transmission unit 3, which emits electromagnetic radiation 4. Radiation 4 is captured by localisation elements 2 and 2′. The localisation elements 2 and 2′ each are comprised of a transponder which is excited by the captured radiation 4 so that the transponder transmits the localisation signal as a (high-frequency) electromagnetic radiation 5 and/or 5′. The localisation signals 5 and 5′ emitted from localisation elements 2 and 2′ are received by three receiving units 6, 7 and 8 arranged at defined positions in space. Receiving units 6, 7 and 8 are connected to an evaluation unit 9 which based on the phase position of the electromagnetic radiation 5 and/or 5′ of the localisation signals at the relevant site of the receiving units 6, 7 and 8 computes the position and/or orientation of the medical instrument 1, i.e. the x-, y- and z-coordinates of the localisation elements 2 and 2′. For the purpose of calibrating a calibrating point 10 is predefined in the coordinate origin. For calibration, the instrument 1 is properly positioned and oriented in such a manner that its tip is located at the calibrating point 10, with instrument 1 having a defined position in space. The phase relation of localisation signal 5 and/or 5′ detected by means of receiving units 6, 7 and 8 during calibration is saved by means of evaluation unit 9. In the further position determination, the evaluation unit 9 puts the signals received from receiving units 6, 7 and 8 into a relationship to the saved calibration data so that the positions of the localisation element 2 and 2′ can be determined in relation to the coordinate origin.

Furthermore, FIG. 1 shows an additional localisation element 2 pertaining to the system. The localisation element 2″ is not arranged at the medical instrument 1. It can be affixed by means of an adhesive connection in detachable arrangement on the skin surface of a patient. The transponder of the additional localisation element 2″ is integrated in a self-adhesive tissue or foil strip like in a conventional plaster. Through the radiation 4 emitted from the transmission unit 3, the transponder of the additional localisation element 2″ is also excited so that it emits a localisation signal 5″. Based on signal 5″, which is also received by means of detection units 6, 7 and 8, the evaluation unit 9 determines the position of the additional localisation element 2″. Thus it is rendered possible to consider the position of a patient as well as the movements of a patient when performing an intervention by means of medical instrument 1.

The following table gives a summarized view of the attenuation values for signal transmission between transmission unit 3, localisation elements 2, 2′, 2″ and receiving units 6, 7, 8 depending on the distance d between transmitter and receiver for various typical RFID transmission frequencies including the relevant wavelengths. The assumption taken on the transmission side is an antenna gain of 1.64 (Dipol) and on the receiver side it is an antenna gain of 1.0. Besides, the table shows the reachable spaces at various frequencies.

| | | | | | 868 MHz | | 915 MHz | | | | | |
|---|---|---|---|---|---|---|---|---|---|---|---|---|
| Distance d | 13.56 MHz | | 433 MHz | | (EU) | | (US) | | 2.45 GHz | | 5.8 GHz | |
| 0.3 m | — | | 12.6 | dB | 18.6 | dB | 19.0 | dB | 27.6 | dB | 35.1 | dB |
| 1 m | — | | 23.0 | dB | 29.0 | dB | 29.5 | dB | 38.0 | dB | 45.5 | dB |
| 2 m | — | | 29.0 | dB | 35.1 | dB | 35.5 | dB | 44.1 | dB | 51.6 | dB |
| 3 m | 2.4 | dB | 32.6 | dB | 38.6 | dB | 39.0 | dB | 47.6 | dB | 55.1 | dB |
| 10 m | 12.9 | dB | 43.0 | dB | 49.0 | dB | 49.5 | dB | 58.0 | dB | 65.6 | dB |
| Reachable | 0-80 | cm | 0-2 | m | 0-5 | m | 0-5 | m | 0-100 | m | 0-5 | km |
| space: | | | | | | | | | | | | |
| Wavelength: | 23 | m | 69.2 | cm | 34.5 | cm | 32.5 | cm | 12.24 | cm | 5.17 | cm |

The table shows that the application of the 433 MHz frequency range with a working range of approx. 70 cm×70 cm×70 cm within space lends itself suitable, whereas the calibration point 10 should maximally be 2 m away from the transmission and/or receiving unit. As described before, the position determination is expediently made based on the phase relation of the localisation signals 5, 5′ and 5″. With a frequency of 433 MHz a phase difference of 1° corresponds to a distance of 1.92 mm. Accordingly, with a desired spatial resolution of 1.92 mm the resolution in determining the phase relation must at least be equal to 1°. Conversely, if a frequency of 5.8 GHz is applied, a spatial resolution of 0.14 mm can already be achieved with a phase resolution of 1. To achieve the highest possible resolution, the transponders of the localisation elements 2, 2′ and 2″ are expediently so arranged that these generate the localisation signals 5, 5′ and 5″ at two or more different frequencies. Thereby a position determination based on the phase relation can be achieved with adequate accuracy. By using low frequencies, the position can initially be determined roughly, though unambiguously. Low frequencies result in a comparably large spatial working range within which the determination of the position can be performed. By using several frequencies, a clear-cut unambiguous determination of the position based on the phase relation is possible while achieving utmost accuracy at the same time.

FIG. 2 sows an intravascular catheter 1 configured in accordance with the invention. Catheter 1 is guided by means of a guidance wire 11 within a blood vessel. Catheter 1 is equipped with a localisation element. In accordance with the invention, the localisation element is equipped with a transponder including a circuit 12 and an antenna 13. The antenna 13 is wound of a thin wire in the longitudinal direction of catheter 1 and connected to the circuit 12. The circuit 12 is an integrated semiconductor chip. By means of antenna 13 the electromagnetic radiation emitted by transmission unit 3 is received. It induces an induction current in antenna 13. The power supply to the circuit 12 is given through this induction current. Hence, with the embodiment shown in FIG. 2, a passive transponder is utilized. For a permanent energy supply to circuit 12 it is connected to a capacitor 14 which is charged by the induction current generated in the antenna 13. The capacitor 14 therefore ensures the function of the transponder even if the induction current generated in the antenna 13 is insufficient for a continuous energy supply. The circuit 12 is activated by the electromagnetic radiation received via antenna 13 and thus excited to emit a localisation signal as electromagnetic radiation via antenna 13. This is accomplished in that the circuit 12 causes a load modulation of the electromagnetic field received by means of antenna 13. The circuit 12 is furthermore linked to a sensor element 15 integrated in the catheter 1, for example to a temperature sensor. The circuit 12 of the transponder transmits the sensor signal of the sensor element 15 as a digital signal via antenna 13. This allows for a wireless determination of the temperature at the relevant site of the tip of catheter 1.

1. A system for determining the spatial position and/or orientation of a medical instrument (1), comprised of a transmission unit (3) emitting an electromagnetic radiation (4), at least one localisation element (2) arranged at a medical instrument (1) which receives the electromagnetic radiation (4) emitted from transmission unit (3) and generates a localisation signal (5), and comprised of an evaluation unit (9), which determines the position and/or orientation of the medical instrument (1) by evaluating the localisation signal (5), wherein the localisation element (2) is comprised of a transponder which is comprised of an antenna (13) and a circuit (12) connected to the antenna (13) for receiving and transmitting electromagnetic radiation, with said circuit (12) being excitable through the electromagnetic radiation (4) from the transmission unit (3) received via the antenna, in such a manner that it emits the localisation signal (5) as electromagnetic radiation through antenna (13).

2. A system as defined in claim 1, wherein the transponder is configured as a passive transponder, with the power supply to said circuit (12) being provided by the induction current generated on reception of the electromagnetic radiation (4) emitted from the transmission unit (3).

3. A system as defined in claim 2, wherein the transponder for power supply to the circuit (12) is comprised of a capacitor (14) which is charged by the induction current generated in the antenna (13).

4. A system as defined in claim 1, wherein the transponder is configured as an active transponder, with a battery being provided for power supply to said circuit (12).

5. A system as defined in claim 1, wherein the frequency of the electromagnetic radiation of the localisation signal (5) differs from the frequency of the electromagnetic radiation (4) emitted from the transmission unit (3).

6. A system as defined in claim 1, wherein the circuit (12) is provided to generate the localisation signal by modulation of the electromagnetic radiation (4) emitted from the transmission unit (3).

7. A system as defined in claim 1, comprising at least one receiving unit (6, 7, 8) connected to the evaluation unit (9), with the evaluation unit (9) being properly provided for determining the position and/or orientation of the medical instrument (1) based on the phase relation of the electromagnetic radiation of the localisation signal (5) at the relevant site of the receiving unit (6, 7, 8).

8. A system as defined in claim 1, wherein the circuit (12) is provided for generating the localisation signal at two or more different frequencies.

9. A system as defined in claim 1, wherein the transponder is connected to at least one sensor element (15), with the circuit (12) of the transponder being properly provided to emit the sensor signal of the sensor element (15) as electromagnetic radiation via the antenna (13) of the transponder.

10. A system as defined in claim 9, wherein the sensor element (15) is a temperature sensor, pressure sensor, pH sensor or position sensor integrated into the medical instrument (1).

11. A system as defined in claim 1, wherein the medical instrument (1) is an intravascular catheter, a guidance wire or a biopsy needle.

12. A system as defined in claim 1, wherein the transponder is an RFID tag.

13. A system as defined in claim 1, wherein at least two localisation elements (2, 2′) with two transponders allocated to them are arranged at the medical instrument (1).

14. A system as defined in claim 1, comprising at least one additional localisation element (2″) not arranged at the medical instrument (1) with a transponder allocated to it which can be affixed in detachable arrangement at a patient's body.

15. A system as defined in claim 14, wherein the additional localisation element (2″) can be affixed by means of a glued, adhesive or suction disk connection in detachable arrangement on a patient's skin surface.

16. A system as defined in claim 14, wherein the transponder of the additional localisation element (2″) is integrated in a self-adhesive foil or tissue strip.

17. A system as defined in claim 1, wherein the transmission unit (3) is the transmission unit of an MR device which is comprised of a transmission/receiving antenna (coil) to generate a high-frequency electromagnetic field in the investigation volume of the MR device.

18. A system as defined in claim 17, wherein the transponder is configured as a passive transponder, with the power supply to the circuit (12) being provided by the induction current generated in the antenna (13) on reception of the high-frequency electromagnetic field during the MR imaging.

19. A system as defined in claim 17, wherein the evaluation unit (9) is linked to the MR device, with the determination of the position and/or orientation of the medical instrument (1) being effected based on the localisation signal (5) received via the transmission/receiving antenna (coil) of the MR device.

20. A system as defined in claim 19, wherein the evaluation unit (9) to determine the position and/or orientation of the medical instrument (1) based on the phase relation of the electromagnetic radiation of the localisation signal (5) is provided at the site of the transmission/receiving antenna of the MR device.

21. A system as defined in claim 1, wherein the evaluation unit (9) is provided for selecting valid position and/or orientation data from a plurality of position and/or orientation data redundantly determined from several localisation signals (5).

22. A system as defined in claim 21, wherein the localisation element (2) is comprised of a plurality of transponders which can be excited in parallel or consecutively for transmitting localisation signals (5).

23. A system as defined in claim 22, wherein the transponders are configured to generate localisation signals (5) at different frequencies each.

24. A system as defined in claim 21, wherein several localisation elements (2) are arranged at the medical instrument (1) to generate redundant localisation signals (5).

25. A medical instrument, more particularly an intravascular catheter (1), guidance wire or biopsy needle, wherein at least one transponder is integrated into the instrument (1), which is comprised of an antenna (13) and a circuit (12) connected to the antenna for receiving and transmitting electromagnetic radiation, with the circuit being excitable through electromagnetic radiation (4) received via the antenna to transmit electromagnetic radiation (5).

26. An instrument as defined in claim 25, wherein the transponder is connected to at least one sensor element (15), with the circuit (12) of the transponder being so provided that it emits the sensor signal of the sensor element (15) as electromagnetic radiation via the antenna (13) of the transponder.

27. An instrument as defined in claim 26, wherein the sensor element (15) is a temperature sensor, a pressure sensor, a pH sensor or a position sensor.

28. An instrument as defined in claim 25, wherein the transponder is an active or a passive RFID tag.

29. An instrument as defined in claim 25, wherein the circuit (12) of the transponder is comprised of a data memory in which identification data can be saved, and that the circuit (12) is properly provided to transmit identification data as electromagnetic radiation via the antenna (13).

30. An instrument as defined in claim 25, wherein at least two transponders are integrated in the instrument (1).

31. A use of an RFID tag for integration into a medical instrument (1) for the purpose of determining the spatial position and/or orientation of the medical instrument (1).

32. A use of an RFID tag for integration into a self-adhesive foil or tissue strip for detachable affixing on a patient's skin surface.

33. A use of an RFID tag for transmission of sensor signals from a sensor element (15) integrated into a medical instrument (1) or implant.

34. A use as defined in claim 33, with the sensor element being a temperature sensor, a pressure sensor, a pH sensor or a position sensor.

35. A method for determining the spatial position and/or orientation of a medical instrument (1), wherein electromagnetic radiation (4) is emitted by means of a transmission unit (3) which is received by at least one localisation element (2) arranged at the medical instrument (1), whereupon the localisation element (2) generates a localisation signal (5) and wherein by means of an evaluation unit (9) the position and/or orientation of the medical instrument (1) is determined by evaluating the localisation signal (5), wherein the localisation element (2) is comprised of a transponder which is comprised of an antenna (13) and a circuit (12) connected to the antenna (13) for receiving and transmitting electromagnetic radiation, with said circuit (12) being excited by the electromagnetic radiation (4) from the transmission unit (3) received via the antenna, whereupon it emits the localisation signal (5) as electromagnetic radiation via the antenna (13).

36. A method as defined in claim 35, wherein the position and/or orientation of the medical instrument (1) is determined based on the phase relation of the electromagnetic radiation of the localisation signal (5) at the site of at least one receiving unit (6, 7, 8) connected to the evaluation unit (9).

37. A method as defined in claim 35, wherein the localisation signal (5) is generated by means of the transponder at two or more different frequencies.

38. A method as defined in claim 35, wherein the medical instrument (1) is an intravascular catheter, a guidance wire or a biopsy needle.

39. A method as defined in claim 35, wherein the transponder is an RFID tag.

40. A method as defined in claim 35, wherein at least two localisation elements (2, 2′) including their relevant transponders allocated to them are arranged at the medical instrument (1), with the orientation of the medical instrument (1) being determined from the localisation signals (5, 5′) of the at least two localisation elements (2, 2′).

41. A method as defined in claim 35, wherein valid position and/or orientation data are selected from a plurality of position and/or orientation data redundantly determined from several localisation signals (5).

42. A method as defined in claim 41, wherein the localisation element (2) is comprised of a plurality of transponders which are excited in parallel or consecutively for the transmission of localisation signals (5).

43. A method as defined in claim 42, wherein the transponders emit localisation signals (5) at different frequencies each.

44. A method as defined in claim 41, wherein several localisation element (2) are arranged at the medical instrument (1) which generate redundant localisation signals (5).
